(19) United States
(12) Patent Application Publication
(10) Pub. No.: US 2008/0208053 A1
(43) Pub. Date: Aug. 28, 2008

(54) ULTRASOUND IMAGE DISPLAYING METHOD AND ULTRASOUND DIAGNOSTIC APPARATUS

(76) Inventor: Hiroshi Hashimoto, Tokyo (JP)

Correspondence Address: Patrick W. Rasche;Armstrong Teasdale LLP, Suite 2600, One Metropolitan Square, St. Louis, MO, 63102, US

(21) Appl. No.: 12/034,485

(22) Filed: Feb. 20, 2008

(30) Foreign Application Priority Data
Feb. 23, 2007 (JP) 2007-043100

(51) Int. Cl. A61B 8/14

(52) U.S. Cl. 600/441

(57) ABSTRACT

An ultrasound image displaying method includes creating a 3D image based on B mode data with a high luminance and a low luminance in a B mode image being inverted, creating a color overlaid 3D image by applying colors based on color Doppler mode data only for pixels of the 3D image having a luminance not more than a predetermined value in the B mode image, and displaying the color overlaid 3D image.

### CROSS REFERENCE TO RELATED APPLICATIONS

This application claims the benefit of Japanese Patent Application No. 2007-043100 filed Feb. 23, 2007.

### BACKGROUND OF THE INVENTION

The field of the present invention relates to an ultrasound image displaying method and an ultrasound diagnostic apparatus and, more specifically, to an ultrasound image displaying method and an ultrasound diagnostic apparatus that enable viewing a 3D structure of a vessel and obtaining 3D images by applying color of a color Doppler image.

Conventionally in the art there is known an ultrasound diagnostic apparatus which is capable of creating and displaying a synthetic image by applying a power Doppler image onto an inverted B mode image with the black and white of the B mode image being inverted (for example, WO97/34530 A1).

In the conventional ultrasound diagnostic apparatus disclosed, a 2D image may be obtained in which the blood vessel may be viewed in white on a black background, and the color of power Doppler image is applied.

However, because the image is in 2D, it may be difficult to distinguish a 3D structure such as a blood vessel.

### SUMMARY OF THE INVENTION

In a first aspect, the invention provides an ultrasound image displaying method that includes creating a 3D image based on B mode data with a high luminance and a low luminance in a B mode image being inverted, creating a color overlaid 3D image by applying colors based on color Doppler mode data only for pixels of the 3D image having a luminance not more than a predetermined value in the B mode image, and displaying the color overlaid 3D image.

In accordance with the ultrasound image displaying method of the first aspect described above, a 3D image with the luminance of the B mode image inverted is created so that the 3D structure such as blood vessels and lumens appear in white on the black background so as to facilitate distinguishing the 3D structure. Any color based on the color Doppler mode data is applied only for pixels having a luminance in the B mode image not more than a predetermined value so that the color will not be smudged out of the 3D structure to allow sightly coloring the structure. In addition, the color is applied only to the region of blood flow, so that the blood vessel (with blood flow) will be readily distinguished from a lumen (without blood flow).

In a second aspect, the invention provides an ultrasound image displaying method of the first aspect described above, in which the luminance of each pixel of the color overlaid 3D image is based on the B mode data, and the color thereof is based on the color Doppler mode data.

The ultrasound image displaying method in accordance with the second aspect described above, any information included in the B mode data and the information included in the color Doppler mode data may be exploitable.

In a third aspect, the invention provides an ultrasound image displaying method of the first or the second aspect described above, further comprising the step of shading the 3D image.

The ultrasound image displaying method in accordance with the third aspect described above, a stereoscopic 3D image may be obtained.

In a fourth aspect, the invention provides an ultrasound image displaying method in accordance with the first or the second aspect described above, further comprising the step of shading the color overlaid 3D image.

The ultrasound image displaying method in accordance with the fourth aspect described above, stereoscopic appearance will be obtained when displaying the color overlaid 3D image.

In a fifth aspect, the invention provides an ultrasound image displaying method in which a 3D image is created based on B mode data with a high luminance and a low luminance in the B mode image being inverted. A color overlaid 3D image is then created by applying color based on power Doppler mode data only for pixels in the 3D image having a luminance in the B mode image not more than a predetermined value. The color overlaid 3D image is then displayed.

In the ultrasound image displaying method in accordance with the fifth aspect described above, a 3D image is created with the high and low of the luminance in the B mode image being inverted, so that the 3D structure such as vessels and lumens appear in white on a black background to facilitate distinguishing the 3D structure. Any color is applied based on the power Doppler mode data only for pixels having a luminance in the B mode image not more than a predetermined value so that the color will not be smudged out of the 3D structure to allow sightly coloring the structure. In addition, the color is applied only to the region of blood flow, so that the blood vessel (with blood flow) may be readily distinguished from a lumen (without blood flow).

In a sixth aspect, the invention provides an ultrasound image displaying method in accordance with the fifth aspect described above, in which the luminance of each pixel of the color overlaid 3D image is based on the B mode data, and the color thereof is based on the power Doppler mode data.

The ultrasound image displaying method in accordance with the sixth aspect described above, any information included in the B mode data and the information included in the power Doppler mode data may be exploitable.

In a seventh aspect, the invention provides an ultrasound image displaying method in accordance with the fifth or the sixth aspect, further comprising the step of shading the 3D image.

In accordance with the ultrasound image displaying method of the seventh aspect described above, a stereoscopic 3D image may be obtained.

In an eighth aspect, the invention provides an ultrasound image displaying method in accordance with the fifth or the sixth aspect described above, further comprising the step of shading the color overlaid 3D image.

In accordance with the ultrasound image displaying method of the eighth aspect described above, a stereoscopic appearance will be obtained when displaying the color overlaid 3D image.

In a ninth aspect, the invention provides an ultrasound diagnostic apparatus comprising: that includes a 3D image creating device for creating a 3D image based on B mode data with a high luminance and a low luminance in a B mode image being inverted; a inverted. The apparatus also includes a color overlaid 3D image creating device for creating a color overlaid 3D image by applying color based on color Doppler mode data only for pixels in the 3D image having a luminance not more than a predetermined value in the B mode image; image, and an image display device for displaying the color overlaid 3D image.

In accordance with the ultrasound diagnostic apparatus of the ninth aspect described above, a 3D image is created with the high luminance and the low luminance in B mode image being inverted, so that the 3D structure such as blood vessels and lumens will be appeared appear in white on a black background so as to facilitate distinguishing the 3D structure. Also, any color based on the color Doppler mode data is applied only for pixels having a luminance in the B mode image not more than a predetermined value so that the color will not be smudged out of the 3D structure and to allow sightly coloring the structure. In addition, the color is applied only to the region of blood flow, so that the blood vessel (with blood flow) may be readily distinguished from a lumen (without blood flow).

In a tenth aspect, the invention provides an ultrasound diagnostic apparatus in accordance with the ninth aspect described above, in which the luminance of each pixels in the color overlaid 3D image is based on the B mode data, and the color thereof is based on the color Doppler mode data.

The ultrasound diagnostic apparatus in accordance with the tenth aspect described above, any information included in the B mode data and the information included in the color Doppler mode data may be exploitable.

In an eleventh aspect, the invention provides an ultrasound diagnostic apparatus in accordance with the ninth or the tenth aspect, in which the 3D image creating device applies shading to the 3D image.

A stereoscopic 3D image may be obtained by using the ultrasound diagnostic apparatus described above in accordance with the eleventh aspect.

In a twelfth aspect, the invention provides an ultrasound diagnostic apparatus in accordance with the ninth and the tenth aspect, in which the 3D image creating device applies shading to the color overlaid 3D image.

The ultrasound diagnostic apparatus in accordance with the twelfth aspect described above a stereoscopic 3D appearance will be obtained when displaying a color overlaid 3D image.

In a thirteenth aspect, the invention provides an ultrasound diagnostic apparatus that includes a 3D image creating device for creating a 3D image with a high luminance and a low luminance in a B mode image being inverted based on B mode data. The apparatus also includes a color overlaid 3D image creating device for creating a color overlaid 3D image by applying color based on power Doppler mode data only for pixels in the 3D image with a luminance in the B mode image not more than a predetermined value, and an image display device for displaying the color overlaid 3D image.

In the ultrasound diagnostic apparatus described above in accordance with the thirteenth aspect, a 3D image is created with a high luminance and a low luminance in a B mode image being inverted, so that the 3D structure such as vessels and lumens appear in white on a black background to facilitate distinguishing the 3D structure. Also, any color based on the power Doppler mode data is applied only for pixels having a luminance in the B mode image not more than a predetermined value so that the color will not be smudged out of the 3D structure and to allow coloring the structure. In addition, the color is applied only to the region of blood flow, so that the blood vessel (with blood flow) may be readily distinguished from a lumen (without blood flow).

In a fourteenth aspect, the invention provides an ultrasound diagnostic apparatus in accordance with the thirteenth aspect, in which the luminance of each pixels of the color overlaid 3D image is based on the B mode data, and the color thereof is based on the power Doppler mode data.

The ultrasound diagnostic apparatus in accordance with the fourteenth aspect described above, any information included in the B mode data and the information included in the power Doppler mode data may be exploitable.

In a fifteenth aspect, the invention provides an ultrasound diagnostic apparatus in accordance with the thirteenth or the fourteenth aspect, in which the 3D image creating device applies shading to the 3D image.

A stereoscopic 3D image may be obtained using the ultrasound diagnostic apparatus described above in accordance with the fifteenth aspect.

In a sixteenth aspect, the invention provides an ultrasound diagnostic apparatus in accordance with the thirteenth or the fourteenth aspect, in which the color overlaid 3D image creating device applies shading to the color overlaid 3D image.

The ultrasound diagnostic apparatus in accordance with the sixteenth aspect described above, a stereoscopic 3D appearance will be obtained when displaying a color overlaid 3D image.

In accordance with the ultrasound image displaying method and the ultrasound diagnostic apparatus of the invention, the three dimensional structure of a blood vessel or a lumen may be visualized. The color will not be smudged out of the three dimensional structure, while sightly well colorized. In addition, the color is applied only to the region of blood flow, so as to facilitate distinguishing the blood vessel (with blood flow) from a lumen (without blood flow).

The ultrasound image displaying method and the ultrasound diagnostic apparatus in accordance with the invention may be used for the diagnosis of a blood vessel and a lumen.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic block diagram illustrating a ultrasound diagnostic apparatus in accordance with the first embodiment of the invention;

FIG. 2 is a flow chart illustrating the color overlaid 3D image display process in accordance with the first embodiment of the invention;

FIG. 3 is a front view, side view, and top view conceptually illustrating the B mode volume data;

FIG. 4 is a front view, side view, and top view conceptually illustrating the color Doppler mode volume data;

FIG. 5 is an example illustrating a 3D image; and

FIG. 6 is an example illustrating a color overlaid 3D image.

### DETAILED DESCRIPTION OF THE INVENTION

The invention will be described in greater details herein below with reference to some embodiments shown in the accompanying drawings. It should be noted here that the disclosed embodiments may not be considered to limit the invention.

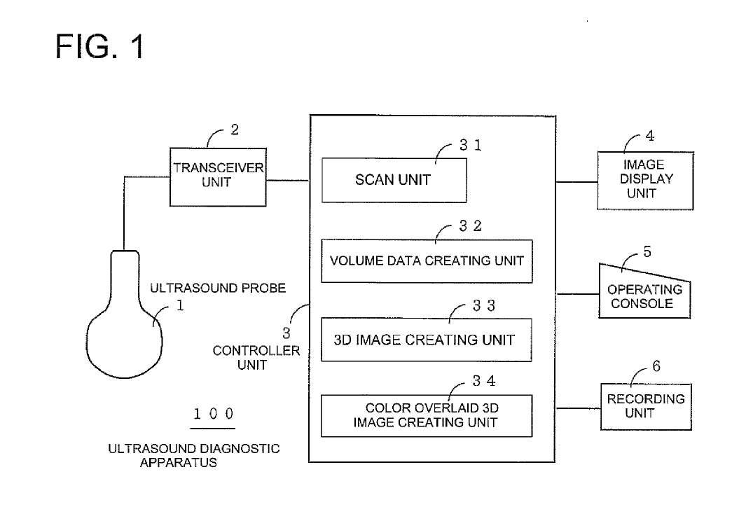

FIG. 1 shows a schematic block diagram of an ultrasound diagnostic apparatus 100 in accordance with the first embodiment.

The ultrasound diagnostic apparatus 100 includes an ultrasound probe 1 capable of 2D scan by electronic scan as well as of 3D scan by electric scan (scan by means of a motor) or electronic scan; a transceiver unit 2 for driving the ultrasound probe 1 to perform 2D scan and 3D scan within the body of an examinee by means of ultrasound beam; a controller unit 3; an image display unit 4 for displaying an ultrasound image; an operating console 5 for inputting an instruction or data by an operator; and a recording unit 6 for recording the ultrasound images.

The controller unit 3 includes a scan unit 31 for controlling the 2D scan and 3D scan; a volume data creating unit 32 for creating volume data; a 3D image creating unit 33 for projecting the volume data in a predetermined projection direction to create a 3D image with the high and low of luminance in the B mode image being inverted; and a color overlaid 3D image creating unit 34 for creating a color overlaid 3D image by applying color based on the color Doppler mode data only for the pixels in a 3D image having the luminance of B mode image not more than a predetermined value.

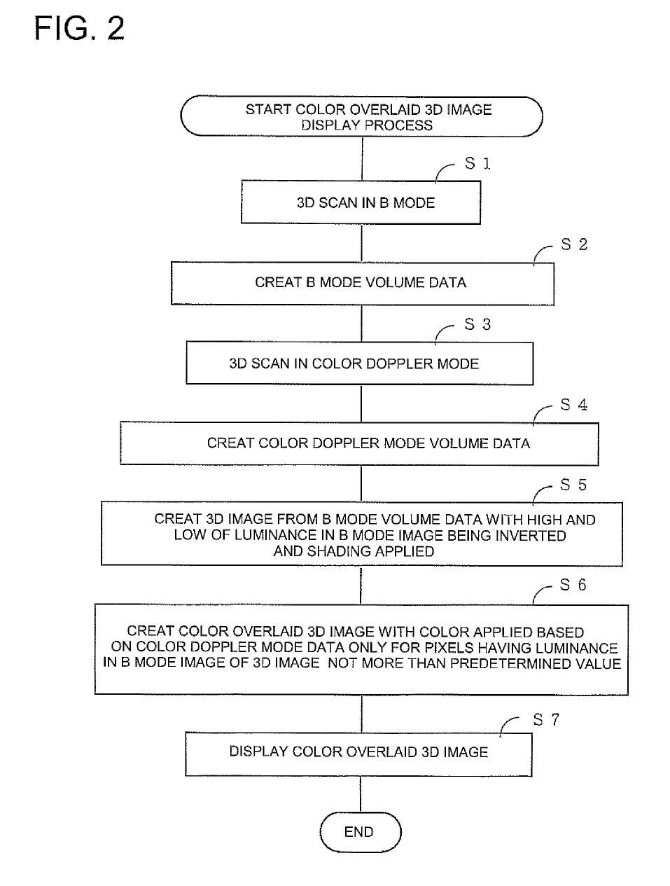

FIG. 2 shows a flow chart illustrating the color overlaid 3D image display process in accordance with the first embodiment.

In step S1, 3D scan is completed in B mode.

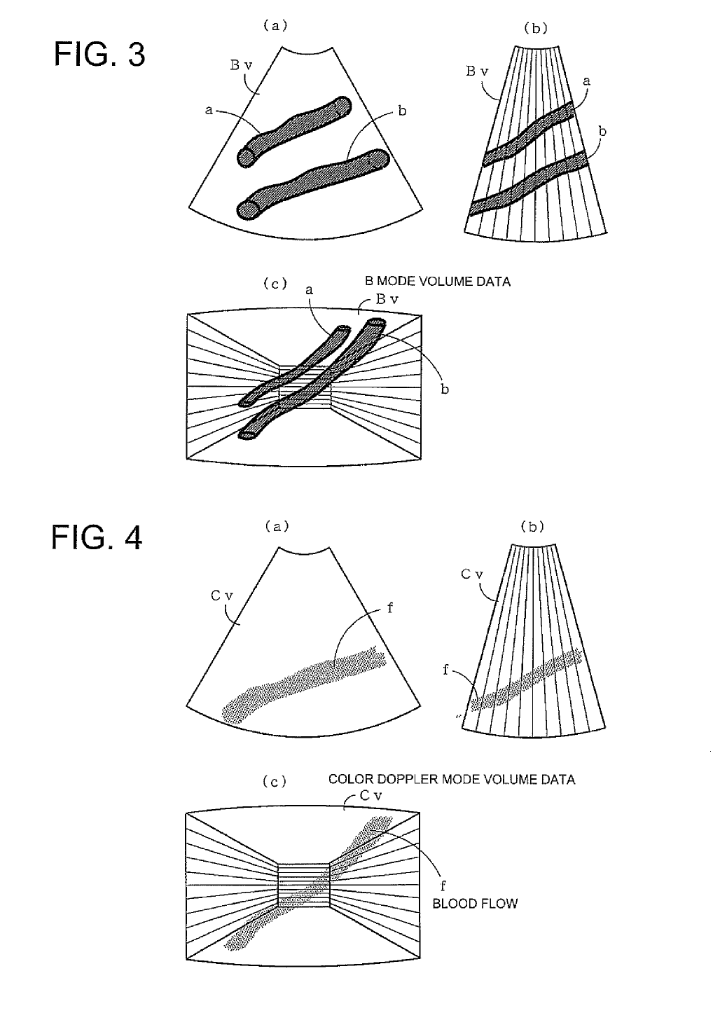

In step S2, B mode volume data By as shown in FIG. 3 is created. In the B mode volume data By the luminance, in the B mode image, of the pixels of the blood vessel or lumen regions a and b will be not more than a predetermined value.

In step S3, 3D scan is completed in color Doppler mode.

In step S4, color Doppler mode volume data Cv as shown in FIG. 4 is created. In the color Doppler mode volume data Cv the region having blood flow f is applied with a color.

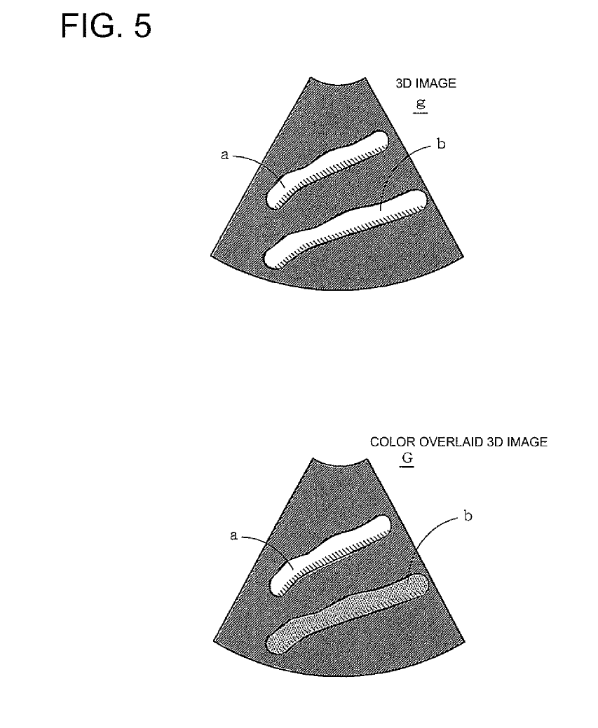

In step S5 a 3D image g is created from the B mode volume data By, as shown in FIG. 5, with the high and low of luminance of the B mode image being inverted and shading applied.

In step S6, by means of MIP process or rendering process, a color overlaid 3D image G will be created with the color based on the color Doppler mode volume data Cv being applied only for the pixels in the region a and b (i.e., pixels having luminance not more than a predetermined value in B mode image) of the 3D image g, as shown in FIG. 6. For example, the luminance of each pixel in the color overlaid 3D image G may be defined according to the B mode volume data By, while the color thereof may be defined according to the color Doppler mode volume data Cv.

In the example shown in FIG. 6, the region a is not colorized since there is no color information in the color Doppler mode volume data Cv. The region b, on the other hand, is colorized since there is color information in the color Doppler mode volume data Cv.

In step S7, the color overlaid 3D image G is displayed.

In accordance with the ultrasound diagnostic apparatus 100 in the first embodiment, one or more of the following effects may be obtained. Regions a and b of the blood vessel and the lumen appear in white on a black background to facilitate visually distinguishing the three dimensional structure. Moreover, the color may not be smudged out of the regions a and b of blood vessel and lumen, so that the image will be colorized. Further, the color will be applied only to the region with blood flow so as to allow identifying that the region a is a lumen without blood flow and that the region b is a blood vessel with blood flow.

In an alternative embodiment, although the shading is applied at the step of 3D image g in the preceding first embodiment, the shading may also be applied at the step of color overlaid 3D image G, or may not be applied at all.

In another alternative embodiment, although the color Doppler mode is cited in the first embodiment, power Doppler mode may also be applied with the invention.

Many widely different embodiments of the invention may be configured without departing from the spirit and the scope of the present invention. It should be understood that the present invention is not limited to the specific embodiments described in the specification, except as defined in the appended claims.

1. An ultrasound image displaying method, comprising:
creating a 3D image based on B mode data with a high luminance and a low luminance in a B mode image being inverted;
creating a color overlaid 3D image by applying colors based on color Doppler mode data only for pixels of said 3D image having a luminance not more than a predetermined value in said B mode image; and
displaying said color overlaid 3D image.

2. An ultrasound image displaying method according to claim 1, wherein:
a luminance of each pixel of said color overlaid 3D image is based on said B mode data, and a color of each pixel of said color overlaid 3D image is based on said color Doppler mode data.

3. An ultrasound image displaying method according to claim 1, further comprising:
shading said 3D image.

4. An ultrasound image displaying method according to claim 1, further comprising:
shading said color overlaid 3D image.

5. An ultrasound image displaying method, comprising:
creating a 3D image based on B mode data with a high luminance and a low luminance in the B mode image being inverted;
creating a color overlaid 3D image by applying color based on power Doppler mode data only for the pixels in said 3D image having a luminance in said B mode image not more than a predetermined value; and
displaying said color overlaid 3D image.

6. An ultrasound image displaying method according to claim 5, wherein:
a luminance of each pixel of said color overlaid 3D image is based on said B mode data, and a color of each pixel of said color overlaid 3D image is based on said power Doppler mode data.

7. An ultrasound image displaying method according to claim 5, further comprising:
shading said 3D image.

8. An ultrasound image displaying method according to claim 5, further comprising:
shading said color overlaid 3D image.

9. An ultrasound diagnostic apparatus, comprising:
a 3D image creating device for creating a 3D image based on B mode data with a high luminance and a low luminance in a B mode image being inverted;
a color overlaid 3D image creating device for creating a color overlaid 3D image by applying color based on color Doppler mode data only for pixels in said 3D image having a luminance not more than a predetermined value in said B mode image; and
an image display device for displaying said color overlaid 3D image.

10. An ultrasound diagnostic apparatus according to claim 9, wherein:
a luminance of each pixel in said color overlaid 3D image is based on said B mode data, and a color of each pixel in said color overlaid 3D image is based on said color Doppler mode data.

11. An ultrasound diagnostic apparatus according to claim 9, wherein:
said 3D image creating device applies shading to said 3D image.

12. An ultrasound diagnostic apparatus according to claim 10, wherein:
said 3D image creating device applies shading to said 3D image.

13. An ultrasound diagnostic apparatus according to claim 9, wherein:
said 3D image creating device applies shading to said color overlaid 3D image.

14. An ultrasound diagnostic apparatus according to claim 10, wherein:
said 3D image creating device applies shading to said color overlaid 3D image.

15. An ultrasound diagnostic apparatus, comprising:
a 3D image creating device for creating a 3D image with a high luminance and a low luminance in a B mode image being inverted based on B mode data;
a color overlaid 3D image creating device for creating a color overlaid 3D image by applying color based on power Doppler mode data only for pixels in said 3D image with a luminance in said B mode image not more than a predetermined value; and
an image display device for displaying said color overlaid 3D image.

16. An ultrasound diagnostic apparatus according to claim 15, wherein:
a luminance of each pixel of said color overlaid 3D image is based on said B mode data, and a color of each pixel of said color overlaid 3D image is based on said power Doppler mode data.

17. An ultrasound diagnostic apparatus according to claim 15, wherein:
said 3D image creating device applies shading to said 3D image.

18. An ultrasound diagnostic apparatus according to claim 16, wherein:
said 3D image creating device applies shading to said 3D image.

19. An ultrasound diagnostic apparatus according to claim 15, wherein:
said color overlaid 3D image creating device applies shading to said color overlaid 3D image.

20. An ultrasound diagnostic apparatus according to claim 16, wherein:
said color overlaid 3D image creating device applies shading to said color overlaid 3D image.
